(19) United States
(12) Patent Application Publication
(10) Pub. No.: US 2012/0276008 A1
(43) Pub. Date: Nov. 1, 2012

(54) RADIOPAQUE INJECTABLE NUCLEUS HYDROGEL COMPOSITIONS

(75) Inventors: Jared W. Walkenhorst, Denver, CO (US); Keith A. Collins, Milford, CT (US); Dennis Y. Lee, Scotch Plains, NJ (US); Thomas G. Wilson, Guilford, CT (US); Thomas J. Douville, West Haven, CT (US); Ulrich Berlemann, Bern (CH)

(73) Assignee: SPINE WAVE, INC., Shelton, CT (US)

(21) Appl. No.: 13/094,512

(22) Filed: Apr. 26, 2011

(51) Int. Cl. A61K 49/00

(52) U.S. Cl. 424/9.1

(57) ABSTRACT

In one embodiment, the invention relates to a composition suitable for preparing a hydrogel useful as replacement material for all or part of a disc nucleus. The composition comprises:
a curable, cross-linkable, non-immunogenic, non-toxic protein comprising a minimum of about 10% and a maximum of about 25% of the hydrogel by weight;
a metal contrast agent comprising a minimum of about 2% and a maximum of about 70% of the hydrogel by weight; and
a crosslinker comprising a minimum of about 0.1% and a maximum of about 10% of the hydrogel by weight;
   wherein the composition has a discernable radiopacity level.

### FIELD OF THE INVENTION

The present invention relates generally to a protein hydrogel composition that is utilized in the treatment of spinal diseases and injuries, and specifically to the restoration of spinal discs. More specifically, the invention contemplates compositions and methods for restoring the normal intervertebral disc space height and for facilitating the introduction of biomaterials for use in the repair and restoration of the intervertebral disc preferably via a percutaneous injection of the hydrogel without an invasive surgical procedure.

### BACKGROUND OF THE INVENTION

There are many causes of disruption or degeneration of the intervertebral disc that can be generally categorized as mechanical, genetic and biochemical. One such cause is the loss of the jelly-like material made in the disc, which is called the disc nucleus pulposus.

Many injectable biomaterials have been developed as a substitute for the disc nucleus pulposus. Such materials include various proteins, including hyaluronic acid, fibrin glue, alginate, elastin-like polypeptides, collagen type I gel and others. See, for example, U.S. Pat. Nos. 5,773,249; 6,380,154 B1; and 6,184,348.

The invasiveness of introducing such materials into a disc may be minimized by injecting the material percutaneously, such as for treatment of degenerative disc disease (DDD). There remains, however, a need for material that is strong and durable, and that can be visualized during the percutaneous procedure.

### SUMMARY OF THE INVENTION

In one embodiment, the invention relates to a composition suitable for preparing a hydrogel useful as replacement material for all or part of a disc nucleus, the composition comprising an aqueous solution and/or suspension that comprises by weight:
a minimum of about 10% and a maximum of about 25% of a curable, cross-linkable, non-immunogenic, non-toxic protein;
a minimum of about 2% and a maximum of about 70% of a metal contrast agent; and
a minimum of about 0.1% and a maximum of about 10% of a crosslinker;
   wherein the hydrogel has a discernable radiopacity level.

In a second embodiment, the invention relates to a composition suitable for preparing a hydrogel useful as replacement material for all or part of a disc nucleus, the composition comprising:
an aqueous solution of a curable, cross-linkable, non-immunogenic, non-toxic protein containing at least one crosslinkable amino acid;
a powder or suspension comprising a metal contrast agent in an amount sufficient to cause the hydrogel to have discernable radiopacity; and
a crosslinker;
   wherein:
(i) the hydrogel has a static compressive modulus of at least about 10 kPa and a dynamic compressive modulus of at least about 10 kPa;
(ii) the static and/or dynamic compressive modulus of the hydrogel may be lower than the static and/or dynamic compressive modulus would have been had the powder or suspension comprising a metal contrast agent not been present in the aqueous solution of the protein; and
   (iii) the amount of the crosslinker is sufficient to restore at least about 100% of the decrease in the static and/or dynamic compressive modulus of the hydrogel caused by the presence of the powder or suspension comprising a metal contrast agent in the aqueous solution of the protein.

In a third embodiment, the invention relates to a method for preparing a hydrogel suitable for use as replacement material for all or part of a disc nucleus, the method comprising:
(a) mixing:
   (1) an aqueous solution of an injectable, curable, cross-linkable, non-immunogenic, non-toxic protein that has at least one crosslinkable amino acid;
   (2) a powder or suspension comprising a metal contrast agent in an amount sufficient to cause the hydrogel to have discernable radiopacity; and
   (3) a crosslinker; and
(b) curing the composition
   wherein:
(i) the hydrogel has a static compressive modulus of at least about 10 kPa and a dynamic compressive modulus of at least about 10 kPa;
(ii) the static and/or dynamic compressive modulus of the hydrogel may be lower than the static and/or dynamic compressive modulus would have been had the powder or suspension comprising a metal contrast agent not been present in the aqueous solution of the protein; and
(iii) the amount of the crosslinker is sufficient to restore, on curing, at least about 100% of the decrease in static and/or dynamic compressive modulus of the hydrogel caused by the presence of the powder or suspension comprising a metal contrast agent in the aqueous solution of the protein.

In a fourth embodiment, the invention relates to a hydrogel suitable for use as replacement material for all or part of a disc nucleus wherein the hydrogel is prepared by curing the composition of the first embodiment.

In a fifth embodiment, the invention relates to a hydrogel suitable for use as replacement material for all or part of a disc nucleus wherein the hydrogel is prepared by curing the composition of the third.

In a sixth embodiment, the invention relates to method for replacing a disc nucleus in a human patient in need thereof, the method comprising:
(a) preparing a liquid composition by mixing:
   (1) an aqueous solution of an injectable, curable, cross-linkable, non-immunogenic, non-toxic protein that has at least one crosslinkable amino acid;
   (2) a powder or suspension comprising a metal contrast agent in an amount sufficient to cause the hydrogel to have discernable radiopacity; and
   (3) a crosslinker;
(b) injecting the liquid composition into a disc of the human patient; and
(c) curing the composition under conditions that cause the liquid composition to become a hydrogel in the disc of the human patient;
wherein:
   (i) the hydrogel has a static compressive modulus of at least about 10 kPa and a dynamic compressive modulus of at least about 10 kPa;
   (ii) the static and/or dynamic compressive modulus of the hydrogel may be lower than the static and/or dynamic compressive modulus would have been had the powder or suspension comprising a metal contrast agent not been present in the aqueous solution of the protein; and
   (iii) the amount of the crosslinker is sufficient to restore, on curing, at least about 100% of the decrease in static and/or dynamic compressive modulus of the hydrogel caused by the presence of the powder or suspension comprising a metal contrast agent in the aqueous solution of the protein.

In a seventh embodiment, the invention relates to kit comprising:
(a) a container comprising a minimum of about 10% and a maximum of about 25% of a curable, cross-linkable, non-immunogenic, non-toxic protein;
(b) a container comprising a minimum of about 2% and a maximum of about 70% of a metal contrast agent; and
(c) a container comprising a minimum of about 0.1% and a maximum of about 10% of a crosslinker;
   wherein:
a liquid composition is formed upon mixing the contents of container (a), container (b) and container (c);
the liquid composition undergoes curing and becomes a hydrogel; and
the hydrogel has a discernable radiopacity level.

In an eighth embodiment, the invention relates to a kit comprising:
(a) an aqueous solution of a curable, cross-linkable, non-immunogenic, non-toxic protein containing at least one crosslinkable amino acid;
(b) a powder or suspension comprising a metal contrast agent in an amount sufficient to cause the hydrogel to have discernable radiopacity; and
(c) a crosslinker;
   wherein:
a liquid composition is formed upon mixing the contents of container (a), container (b) and container (c);
the liquid composition undergoes curing and becomes a hydrogel;
the hydrogel has a discernable radiopacity level;
the hydrogel has a static compressive modulus of at least about 10 kPa and a dynamic compressive modulus of at least about 10 kPa;
the static and/or dynamic compressive modulus of the hydrogel may be lower than the static and/or dynamic compressive modulus would have been had the powder or suspension comprising a metal contrast agent not been present in the aqueous solution of the protein; and
the amount of the crosslinker is sufficient to restore at least about 100% of the decrease in static and/or dynamic compressive modulus of the hydrogel caused by the presence of the powder or suspension comprising a metal contrast agent in the aqueous solution of the protein.

### DETAILED DESCRIPTION OF THE PREFERRED EMBODIMENTS

### Compositions

The compositions of the invention are prepared by mixing together a solution of a protein; a powder or suspension of a metal contrast agent; and a crosslinker. Mixing the solution of a protein and the powder or suspension of a metal contrast agent results in a solution of the protein and a suspension of the metal contrast agent (a solution/suspension).

The inventive compositions are aqueous solutions or suspensions that are useful in preparing a protein hydrogel capable of replacing a portion or substantially all of the natural material in an intervertebral disc nucleus, i.e., the nucleus pulposus. The compositions are injectable, i.e., the compositions can be infused into a damaged disc nucleus from a syringe, pump, or similar device as a liquid by methods known in the art.

As used herein, protein hydrogels are solid networks of protein molecules within a liquid medium. The association between protein chains can be either covalent or non-covalent. The protein hydrogels preferably possess a degree of flexibility similar to that of natural tissue, due to their significant water content.

Intervertebral discs lie between adjacent vertebrae in the spine. Such discs include, for example, lumbar discs, thoracic discs, cervical discs, and sacral discs.

### The Protein

The compositions comprise a protein that can be crosslinked to form a hydrogel. In one embodiment, the hydrogel comprises a minimum of about 10% and a maximum of about 25% of the protein by weight.

In one embodiment, the protein is a protein block copolymer that comprises one, two, three, or four of the following segments:
(i) a segment having at least one sequence comprising the six amino acid repetitive sequence found in naturally occurring silk;
(ii) a segment having at least one sequence comprising the five amino acid repetitive sequence found in naturally occurring elastin;
(iii) a segment having at least one sequence comprising the 3 amino acid repetitive sequence found in naturally occurring collagen; or
(iv) a segment having at least one sequence comprising the 7 amino acid repetitive sequence found in naturally occurring keratin; or
combinations of any two, three, or all four of the above.

In another embodiment, the protein is a protein block copolymer, preferably a block copolymer that comprises the following segments:
(i) a segment having at least 2 sequences, wherein each of the sequences is comprised of the six amino acid repetitive sequence found in naturally occurring silk;
(ii) a segment having at least 2 sequences, wherein each of the sequences is comprised of the five amino acid repetitive sequence found in naturally occurring elastin;
(iii) a segment having at least 2 sequences, wherein each of the sequences is comprised of the 3 amino acid repetitive sequence found in naturally occurring collagen; or
(iv) a segment having at least 2 sequences, wherein each of the sequences is comprised of the 7 amino acid repetitive sequence found in naturally occurring keratin; or
combinations of any two, three, or all four of the above.

Preferably, the protein comprises a total of at least 50 sequences, and a maximum number of 500 sequences.

In addition, the protein comprises at least one amino acid residue that contains a functional group that can be cross linked. Some examples of naturally occurring functional groups include amino groups on lysine and histidine residues, carboxyl groups on aspartate and glutamate residues, guanidino groups on arginine residues, hydroxyl groups on serine and threonine residues, and thiol groups on cysteine residues.

If the protein does not contain a functional group that is available for cross linking, an amino acid residue of the protein, including possibly an amino acid of a sequence described above, is replaced with an amino acid that contains such a functional group. The protein has at least one amino acid that contains a cross-linkable functional group. Preferably, the protein has at least 0.1%, more preferably at least 1%, and most preferably at least about 2% amino acids that contain a cross-linkable functional group. Usually, the protein has no more than 20%, preferably no more than 10%, and more preferably no more than 5% amino acids that contain a cross-linkable functional group.

An example of a sequence of amino acids found in naturally occurring silk (Sk) is GlyAlaGlyAlaGlySer. An example of a sequence of amino acids found in naturally occurring elastin (Es) is GlyValGlyValPro. An example of a sequence of amino acids found in naturally occurring collagen (Cl) is GlyAlaPro. An example of a sequence of amino acids found in naturally occurring keratin (Kr) is LysLeuGluLeuAlaGluAla.

In one aspect, the protein has the formula:

NH₂—(U)ₙ₆—[[B]ₙ₁-[J]ₙ₂]ₙ₃-(Z)ₙ₇—COOH (Formula 1)
wherein:
   U and Z represent amino acid sequences other than Sk, Es, Cl, or Kr, and preferably sequences that are useful in cloning the protein;
   B independently at each position represents Sk, Es, Cl, or Kr;
   J independently at each position represents Sk, Es, Cl, or Kr, but not the sequence in B;
   Sk represents GlyAlaGlyAlaGlySer;
   Es represents GlyValGlyValPro;
   Cl represents GlyAlaPro;
   Kr represents LysLeuGluLeuAlaGluAla; and
   n1 represents 1-12, and more usually 2-6, and more usually 2-4;
   n2 represents 1-16, and more usually 2-12, and more usually 4-8;
   n3 represents 1-30, more usually 4-20, and more usually 12-18;
   n6 and n7 independently represent 0 or 1; and
   wherein at least one of the sequences represented by B or J comprises, or is modified to comprise, at least one crosslinkable amino acid residue, e.g., lysine, histidine, aspartatic acid, glutamic acid, arginine, serine, threonine, or cysteine.

In this specification, the term “amino acid sequences other than Sk, Es, Cl, or Kr that are useful in cloning the protein,” and phrases like it, e.g., U and Z, means the amino acid sequence that is encoded by a nucleotide sequence that is helpful in isolating or purifying the nucleic acid molecule that encodes the protein.

In one embodiment of Formula 1, B represents Sk or Es. In another embodiment, J represents Sk or Es. In a preferred embodiment, B and J represent Sk or Es. In a more preferred embodiment, B represents Sk and J represents Es.

In another aspect of Formula 1, the protein has the formula:

NH₂—(U)ₙ₆-[(Sk)ₙ₃(Es)ₙ₄]ₙ₅—(Z)ₙ₇—COOH
wherein:
   n3 represents 1-12;
   n4 represents 1-16;
   n5 represents 1-30;
   n6 and n7 represent 0 or 1; and
wherein at least one of the sequences represented by Sk or Es is modified to comprise at least one crosslinkable amino acid residue, e.g., lysine, histidine, aspartatic acid, glutamic acid, arginine, serine, threonine, or cysteine.

In a more particular aspect of Formula 1, the protein has the formula:

NH₂—(U)ₙ₆-[(Sk)₂(Es)₈]₁₇—(Z)ₙ₇—COOH

wherein at least one of the sequences represented by Sk or Es is modified to comprise at least one crosslinkable amino acid residue, e.g., lysine, histidine, aspartatic acid, glutamic acid, arginine, serine, threonine, or cysteine. In a preferred embodiment of this aspect, at least one Es segment is replaced by an amino acid sequence selected from the group consisting of GlyXGlyValPro or GlyValGlyXPro wherein X represents lysine, histidine, aspartic acid, glutamic acid, arginine, serine, threonine, and cysteine; and preferably GlyLysGlyValPro or GlyValGlyLysPro.

In an even more particular aspect of Formula 1, the protein has the formula:

NH₂—(U)ₙ₆-[(Sk)₂(Es)₄Es′(Es)₃]₁₇—(Z)ₙ₇—COOH

wherein Es′ represents GlyLysGlyValPro or GlyValGlyLysPro. Preferably, Es′ represents GlyLysGlyValPro (Such protein is known as P27K). The invention also covers proteins having an amino acid sequence that is at least 90% identical to the amino acid sequence of P27K, and preferably at least 95% identical to the amino acid sequence of P27K.

The protein preferably has a minimum molecular weight of at least about 20 kD, generally at least about 30 kD, preferably at least about 50 kD. The maximum molecular weights are usually not more than about 205 kD, more usually not more than about 150 kD, preferably not more than about 100 kD, and even more preferably not more than about 77 kD. The proteins will have at least two functional groups available for crosslinking, more usually at least about four function groups available for crosslinking. The equivalent weight per functional group is generally in the range of about 1 kD to 40 kD, more usually in the range of about 5 kD to 25 kD, preferably in the range of about 7 kD to 10 kD.

### Metal Contrast Agents

The injectable compositions of the present invention further contain a metal contrast agent, suitable for use in imaging methods known in the art in order to provide acceptable visibility in the lumbar spine during injection. The metal contrast agent is non-iodinated, and is useful in distinguishing surrounding tissue that is subjected to detectable forms of radiation such as, for example, X radiation, radioactivity, infrared radiation, ultraviolet radiation, electron or neutron radiation, or a magnetic field. Accordingly, the metal contrast agents are useful in imaging methods such as fluoroscopy, X-ray radiography, computerized tomography (CT), ultrasound, etc.

In one embodiment, the metal contrast agent comprises any metal or metal ion that is useful in distinguishing surrounding tissue that is subjected to detectable forms of radiation, as described above. Some suitable examples of such metals or metal ions include tantalum, tungsten, titanium, gold, platinum, iridium, rhodium, palladium, barium, gadolinium, zirconium, and bismuth.

The metal contrast agent is a base metal or a metal salt. Base metals are preferably used in powder form.

Some examples of useful salts of metal ions include, but are not limited to, metal oxides, carbonates, subcarbonates, sulfates, fluorides, chlorides, or bromides. Base metals, (e.g., metal powders) and metal salts can be in solution or in suspension in a liquid vehicle, preferably water. The water is preferably saline, buffered, and more preferably isotonic, e.g., PBS. The water optionally comprises organic solvents with low toxicity, e.g., ethanol, DMSO. The water may be buffered with any non-toxic buffer, for example, a phosphate buffer, e.g., NaPi buffer; sodium, potassium or calcium acetate/acetic acid; sodium, potassium or calcium tartrate/tartaric acid; or sodium, potassium or calcium citrate/citric acid.

Some preferred contrast agents include the base metal (e.g., powder) or salt forms (e.g., oxides, carbonates, sulfates, fluorides, chlorides, or bromides, preferably oxides, carbonates, or sulfates) of tantalum, tungsten, titanium, gold, platinum, iridium, rhodium, palladium, barium, and bismuth. Some specifically preferred contrast agents include zirconium oxide; barium sulfate; bismuth oxide, and bismuth subcarbonate.

A powder or suspension comprising a metal contrast agent is added to the protein solution in an amount sufficient to make the composition radiopaque. In one embodiment, there are substantially no covalent bonds between an atom of the metal contrast agent and an atom of the protein in the hydrogel.

The hydrogels of the present invention are considered to be radiopaque when radiopacity is at a discernable level, i.e., a level that is detectable by suitable imaging methods, e.g., fluoroscopy, CT, X-ray, etc. As used herein, terms such as “radiopacity,” “radiopaque” and the like refer to the relative inability of electromagnetic radiation, particularly X-rays, to pass through a particular material. For example, the metal contrast agents of the invention inhibit the passage of electromagnetic radiation through a hydrogel containing them sufficiently to cause a relatively opaque, appearance in radiographic imaging.

In one embodiment, the hydrogel composition comprises a metal contrast agent in an amount that gives the composition a discernable radiopacity level. Preferably, the contrast agent comprises a minimum of about 2%, preferably of about 5%; and a maximum of about 70%, preferably of about 35% of the hydrogel by weight.

In a preferred embodiment, radiopacity may be permanent or transient. As used herein, transient refers to a state of being brief and short-lived. For example, radiopacity is transient if the hydrogel maintains radiopacity for a maximum of usually not more than 30 days, more usually not more than 14 days, preferably not more than 7 days and more preferably not more than 2 days.

### Crosslinker

The crosslinker, sometimes referred to below as a crosslinking agent, is a molecule that has at least two groups, each of which is able to form a covalent bond with a crosslinkable functional group on the protein, e.g., an amino, carboxyl, guanidino, hydroxyl, or thio group. The functional groups for crosslinking may be all the same or combinations of functional groups that are able to form covalent bonds with the functional groups and may include the functional groups, such as amino, e.g. lysine, histidine; carboxyl, e.g. aspartate and glutamate; guanidino, e.g. arginine; hydroxyl, e.g. serine and threonine; and thiol, e.g. cysteine. For example, the crosslinker may have a functional group that reacts with a naturally occurring functional group on an amino acid of the protein.

Various reactive functional groups may be employed on the crosslinker, such as aldehyde, isocyanate, mixed carboxylic acid anhydride, e.g., ethoxycarbonyl anhydride, activated olefin, halo, amino, and the like. By appropriate choice of the functional groups on the protein polymer and the crosslinking agent, rate of reaction and degree of crosslinking can be controlled.

In one embodiment, the crosslinker has the formula: X¹—R—X²; wherein X¹ and X² are functional groups that form covalent bonds with the functional groups of the protein. R represents a saturated or unsaturated hydrocarbyl chain (i.e. alkyl or alkenyl) having a minimum of 2, preferably 3, and more preferably 4 carbon atoms in the chain; and a maximum of 24, preferably a maximum of 12, and more preferably a maximum of 8. The carbon atoms in the hydrocarbyl chain may optionally be replaced by at least one heteroatom; wherein heteroatoms are selected from the group consisting of —O— or —NH₂—; and wherein each heteroatom is separated from each other heteroatom by at least two carbon atoms. In one embodiment, X¹ and X² are ester groups or isocyanate groups.

Various crosslinking agents may be employed, particularly those which have been used previously and have been found to be physiologically acceptable. Some useful functional groups on the crosslinker include, for example dialdehydes, such as glutaraldehyde, activated diolefins, diisocyanates such as, tetramethylene diisocyanate, hexamethylene diisocyanate, octamethylene diisocyanate, acid anhydrides, such as succinic acid dianhydride, ethylene diamine tetraacetic acid dianhydride, diamines, such as hexamethylene diamine, cyclo(L-lysyl-L-lysine) isocyanate groups (e.g., tetramethylene diisocyanate, hexamethylene diisocyanate, octamethylene diisocyanate) aldehyde groups (glutaraldehyde); acid anhydrides. Preferably, the functionality is amino. (including guanidine), etc. The crosslinking agent may also contain unsymmetrical functional groups, for example, activated olefin aldehydes, e.g. acrolein and quinoid aldehydes, activated halocarboxylic acid anhydride, and the like. The crosslinking agents will usually be commercially available or may be readily synthesized in accordance with conventional methods known in the art, either prior to application of the adhesive or by synthesis in situ.

Thus, depending upon the function groups on the protein and on the crosslinking agent, one can form amides, imines, ureas, esters, ethers, urethanes, thio ethers, disulfides, and the like.

The crosslinking agent contains two or more functional groups, usually not exceeding four functional groups. The functional groups may be the same or different. The ratio of crosslinking agent to protein will vary widely, depending upon the crosslinking agent, the number of functional groups present on the protein, the desired rate of curing, and the like. Generally, the weight ratio of the protein to crosslinking agent will be at least about 1 to 1, usually at least about 10 to 1, and generally at least about 20 to 1; and not greater than about 250 to 1, usually not greater than about 150 to 1, and generally not greater than about 50 to 1. Considerations in selecting the protein-crosslinking agent equivalent ratio will be the rate of setup, reactivity of the crosslinking agent, relative solubility of the crosslinking agent in the mixture, physiological properties of the crosslinking agent, desired degree of stability of the crosslinked product, and the like.

The crosslinker may be added to the composition neat, in liquid suspension, or in solution. The liquid vehicle of the suspension is water, an organic solvent that is non-toxic to humans, or a combination thereof. The water is preferably saline, and more preferably isotonic, e.g., PBS. Suitable organic solvents include ethanol and DMSO.

### Concentration of the Crosslinker

The presence of the powder or suspension comprising the metal contrast agent in the protein solution causes a dilution of the protein, and may lead to a corresponding loss of mechanical strength of the resultant hydrogel. Accordingly, the static and/or dynamic compressive modulus of the hydrogel may be lower than the static and/or dynamic compressive modulus would have been had the powder or suspension comprising a metal contrast agent not been present in the aqueous solution of the protein. In such cases, the compositions of the invention comprise a sufficient amount of a crosslinker to restore at least about 100%, and preferably at least about 150%, of the decrease in static and/or dynamic compressive modulus. If there is no loss in the modulus, the concentration of the crosslinker need not be adjusted.

Increasing the concentration of the crosslinker in the composition can be used to offset the reduction in the mechanical strength of hydrogels prepared from diluted protein concentrations caused by the presence of the powder or suspension comprising the metal contrast agent in the aqueous solution of the protein, and/or to improve other material properties of the hydrogels. For example, adhesion of the implant to a collagen substrate is an important component in keeping the implant in place within the disc, and is measured by extrusion resistance testing. Adhesion to collagen may be increased about 30% and the extrusion resistance increased about 100% in accordance with the invention relative to the adhesion and extrusion resistance that would have resulted from preparing the protein hydrogels in the absence of the powder or suspension comprising the metal contrast agent in the aqueous solution of the protein.

Accordingly, the concentration of the crosslinker is important in the present invention, and is based on the crosslinking density required to restore the loss of mechanical strength of the protein hydrogel due to the presence of the powder or suspension of metal contrast agent in the protein solution during preparation of the hydrogel. Typically, the crosslinker is included in an amount of a minimum of about 0.1% and a maximum of about 10% of the hydrogel by weight.

### Curing, Protein Hydrogel, and Administration

When a composition described above is cured, it forms a protein hydrogel. The protein hydrogel preferably has a minimum static and a minimum dynamic compressive modulus each of about 10 kPa, and preferably each about 20 kPa. The maximum static and a dynamic compressive modulus is each about 500 kPa, and preferably each about 300 kPa. Static and Dynamic Compressive Moduli are measures of the mechanical strength of the hydrogels, and represent the ratio of stress to strain in compression under static and vibratory conditions, respectively. (See, for example, Meyers and Chawla (1999): “Mechanical Behavior of Materials,” pp 98-103).

The proteins of the compositions of the invention are cured in the presence of the crosslinker. Protein crosslinking (i.e., curing) causes crosslinking of the protein, and is carried out under conditions that restore at least about 100%, and preferably at least about 150%, of the decrease in static and/or dynamic compressive modulus of the protein hydrogel caused by the presence of the powder or suspension comprising the metal contrast in the solution comprising the protein. Such conditions are known in the art.

Prior to adding the crosslinker, the solution comprising the protein and the powder or suspension comprising the metal contrast agent should be mixed well together. Mixing causes the contrast agent to become evenly interspersed with the protein solution and, therefore, also within the resulting protein hydrogel.

Typically, curing begins with the addition of the crosslinker to the solution/suspension of the protein and the metal contrast agent, preferably at room temperature. The temperature may be increased or decreased to increase or decrease the rate of crosslinking Curing continues after injection into the disc of a patient at the temperature inside the disc.

Addition of the curing agent to the solution/suspension of the protein and the metal contrast agent preferably takes place immediately prior to being injected into the patient, i.e., while the composition is still a liquid. The composition is prepared preferably within 120 seconds, more preferably within 90 seconds, and most preferably within 60 seconds of being injected.

Curing of the protein continues in situ inside the disc, and causes the liquid composition to become a hydrogel. Depending on the crosslinking conditions, such as the concentrations in the composition of the protein and the crosslinker, curing may, for example, be complete in 2-120 minutes, preferably 5-60 minutes, more preferably 30-40 minutes.

After curing, the hydrogels are non-immunogenic and non-toxic.

Methods and instruments for injecting curable biomaterial into a damaged disc in a percutaneous procedure are known in the art. Some examples of suitable methods and instruments are described in published patent application US2006/009851, filed on Jun. 29, 2005, commonly assigned to the same assignee as the present invention. Such methods and instruments are incorporated herein by reference.

The compositions and the protein hydrogels of the invention are non-immunogenic, i.e., the compositions and the protein hydrogels do not cause a significant immune response. The compositions and the protein hydrogels are also “non-toxic,” i.e., the compositions and the protein hydrogels do not injure or cause significant harm to the subject of the nuclear replacement, usually a person or a laboratory animal. If the metal contrast agent is transient, it will contribute less to immunogenicity or toxicity.

The compositions and the protein hydrogels in accordance with the present invention may vary widely as long as the cured hydrogels achieve the claimed criteria. A suitable example of such a composition has, for example, by weight a minimum of about 5%, preferably about 30% water, and a maximum of about 85% water; a minimum of about 10% protein and a maximum of about 25% of protein; a minimum of about 2%, preferably of about 5% and a maximum of about 70%, preferably of about 35% of metal contrast agent; and a minimum of about 0.1% crosslinker and a maximum of about 10% crosslinker. The composition may also include buffer salts and/or other formulation additives, typically in the range of about 0.1%-10% by weight. Such buffer salts and formulation additives may include sodium and/or potassium phosphate, sodium acetate, sodium chloride, tromethamine, sodium citrate, sucrose and mannose.

While the compositions and the protein hydrogels of the present invention have been described herein particularly with respect to percutaneous injection, it should be appreciated that the subject composition may also be used as an injectable composition in non-percutaneous procedures, such as in open surgical procedures for the treatment of degenerative disc diseases (DDD) or as an adjunct to microdiscectomy (AMD) procedures, also described in the aforementioned published application US2006/0009851.

### Kits

Any of the components of the compositions described above, e.g., solution comprising proteins, powder or aqueous suspension comprising metal contrast agents, and crosslinker, may be placed in individual containers, which can then be packaged into kits. A kit contains at least two components, and preferably all three.

In one embodiment, two of the components may be placed in the same container. For example, the solution comprising proteins and powders or aqueous suspension comprising metal contrast agent may be placed in a single container. Alternatively, the powder or aqueous suspension comprising metal contrast agent and the crosslinker may be placed in a single container.

The container will typically be a plastic or glass vial or a plastic or glass syringe. Preferably, the container contains a pre-measured amount of the components, and can be administered to a patient without further measuring. For example, a kit may contain a syringe containing 4 mL of protein solution, a vial containing 300 mg of gold powder, and a vial containing 0.1 mL of hexamethylene diisocyanate.

When a patient is ready to receive a composition of the invention, the components are mixed together and administered as described above.

### EXAMPLES

The following examples are directed to radiopaque injectable nucleus hydrogel compositions of some embodiments of the present invention. “NuCore® polymer” refers to uncrosslinked protein P27K. NuCore® material refers to crosslinked P27K hydrogel.

### Example 1

The mechanical effect on the NuCore® material by the addition of a radiopaque metal powder has been evaluated by fatigue testing and by mechanical characterization testing. The material characteristics of the cylinders with RP powders were compared to cylinders made with regular NuCore® material. The test was conducted after 1 day and 7 days of aging at 37 deg C. The results are shown in Table 1.

TABLE 1
Mechanical characterization (E*) test results of Radiopaque Powder

| | Day 1 | | | |
|---|---|---|---|---|
| | DMC | | Day 7 | |
| | (kPa) | | [symbol] | |
| S2 verif (control upper rate) | 77 | 86.4% | 105 | 65.5% |
| S2 verif (control lower rate) | 79 | 83.2% | 96 | 72.5% |
| Bismuth Oxide 345 mg | 83 | 81.5% | 117 | 59.6% |
| Tantalum 300 mg | 79 | 80.1% | 110 | 60.3% |
| Barium Sulfate 515 mg | 58 | 100%* | 102 | 66.7% |
| Zirconium Oxide 380 mg | 87 | 82.3% | 124 | 60.8% |
| Gold 300 mg | 75 | 79.6% | 106 | 61.4% |

*Tested at 0-day so E* is artificially low in comparison [symbol]
indicates data missing or illegible when filed

It can be seen from the data in Table 1 that the addition of the RP powders does not appear to negatively affect the mechanical performance of the NuCore® material.

Six NuCore® material test samples were created with various RP additives. One sample was created with regular NuCore® material. Each of the other five test samples was created by mixing in the following quantity of powder with 4.0 mL of P27K protein prior to crosslinking: either 300 mg Gold, 300 mg Tantalum, 350 mg Bismuth Oxide, 380 mg Zirconium Oxide, or 515 mg Barium Sulfate. 40 μL hexamethylene diisocyanate (crosslinker) was then added to each protein+metal powder mixture, and the resultant solution well mixed. This mixture was injected into a silicone disc model and cured in situ. Each resultant test specimen was cycled between −300 N and −3000 N at a rate of 3 Hz for 1 million cycles. After testing, each specimen was sectioned and the condition of the NuCore® hydrogel was observed.

The radiopaque additives did not appear to negatively affect the fatigue properties of the NuCore material in the silicone disc model.

### Examples 1A-1D

Radiopaque injectable nucleus hydrogel compositions are prepared in accordance with the compositions indicated in Examples 1A-1D, with bismuth oxide, tantalum, barium sulfate, zirconium oxide and gold as the contrast agents; hexamethylene diisocyanate as the crosslinker; and sodium phosphate as the salt buffer.

### Composition of Example 1A

| Component | Weight % |
|---|---|
| P27K | 17.7 |
| bismuth oxide | 7.5 |
| hexamethylene diisocyanate | 0.9 |
| sodium phosphate | 1 |
| Water | Remainder |

### Composition of Examples 1B

| Component | Weight % |
|---|---|
| P27K | 17.9 |
| tantalum | 6.5 |
| hexamethylene diisocyanate | 0.9 |
| salts sodium phosphate | 1 |
| Water | Remainder |

### Composition of Examples 1C

| Component | Weight % |
|---|---|
| P27K | 17.1 |
| barium sulfate | 10.7 |
| hexamethylene diisocyanate | 0.9 |
| sodium phosphate | 1 |
| Water | Remainder |

### Composition of Examples 1D

| Component | Weight % |
|---|---|
| P27K | 17.5 |
| zirconium oxide | 8.1 |
| hexamethylene diisocyanate | 0.9 |
| sodium phosphate | 1 |
| Water | Remainder |

### Examples 2A-2E

Radiopaque injectable nucleus hydrogel compositions are prepared in accordance with the compositions indicated in Examples 2A-2E with metal contrast agents; hydrogenated diphenylmethane diisocyanate, tolylene diisocyanate, and glutaraldehyde as the crosslinker; and sodium citrate, sodium acetate, and tromethamine as the buffer salt.

### Composition of Example 2A

| Component | Weight % |
|---|---|
| P27K | 17 |
| gold powder | 6.4 |
| hydrogenated diphenylmethane diisocyanate | 1.5 |
| sodium citrate | 2 |
| Water | Remainder |

### Composition of Examples 2B

| Component | Weight % |
|---|---|
| P27K | 17.5 |
| tantalum powder | 6.4 |
| tolylene diisocyanate | 2.6 |
| sodium acetate | 0.7 |
| Water | Remainder |

### Composition of Examples 2C

| Component | Weight % |
|---|---|
| P27K | 16.5 |
| barium sulfate | 10.5 |
| hydrogenated diphenylmethane diisocyanate | 3.9 |
| sodium citrate | 2.3 |
| Water | Remainder |

### Composition of Examples 2D

| Component | Weight % |
|---|---|
| P27K | 16.8 |
| barium sulfate | 10.6 |
| tolylene diisocyanate | 2.5 |
| sodium acetate | 0.7 |
| Water | Remainder |

### Composition of Examples 2E

| Component | Weight % |
|---|---|
| P27K | 18 |
| gold powder | 5 |
| glutaraldehyde | 0.6 |
| tromethamine | 1 |
| Water | Remainder |

What is claimed is:

1. A composition suitable for preparing a hydrogel useful as replacement material for all or part of a disc nucleus, the composition comprising an aqueous solution or suspension that comprises by weight:
a minimum of about 10% and a maximum of about 25% of a curable, cross-linkable, non-immunogenic, non-toxic protein;
a minimum of about 2% and a maximum of about 70% of a metal contrast agent; and
a minimum of about 0.1% and a maximum of about 10% of a crosslinker;
wherein the hydrogel has a discernable radiopacity level.

2. A composition suitable for preparing a hydrogel useful as replacement material for all or part of a disc nucleus, the composition comprising:
an aqueous solution of a curable, cross-linkable, non-immunogenic, non-toxic protein containing at least one crosslinkable amino acid;
a powder or suspension comprising a metal contrast agent in an amount sufficient to cause the hydrogel to have discernable radiopacity; and
a crosslinker;
wherein:
(i) the hydrogel has a static compressive modulus of at least about 10 kPa and a dynamic compressive modulus of at least about 10 kPa;
(ii) the static and/or dynamic compressive modulus of the hydrogel may be lower than the static and/or dynamic compressive modulus would have been had the powder or suspension comprising a metal contrast agent not been present in the aqueous solution of the protein; and
(iii) the amount of the crosslinker is sufficient to restore at least about 100% of the decrease in the static and/or dynamic compressive modulus of the hydrogel caused by the presence of the powder or suspension comprising a metal contrast agent in the aqueous solution of the protein.

3. The composition of claim 2, wherein the protein comprises:
a segment having at least one sequence comprising the six amino acid repetitive sequence found in naturally occurring silk;
a segment having at least one sequence comprising the five amino acid repetitive sequence found in naturally occurring elastin;
a segment having at least one sequence comprising the three amino acid repetitive sequence found in naturally occurring collagen;
a segment having at least one sequence comprising the seven amino acid repetitive sequence found in naturally occurring keratin; or
combinations thereof;
wherein at least one amino acid residue in the at least one sequence is replaced by a lysine, histidine, serine, aspartaic acid, glutamic acid, arginine, threonine, or cysteine residue.

4. The composition of claim 2, wherein the protein is a block copolymer that comprises:
a segment having at least 2 sequences, wherein each of the sequences is comprised of the six amino acid repetitive sequence found in naturally occurring silk;
a segment having at least 2 sequences, wherein each of the sequences is comprised of the five amino acid repetitive sequence found in naturally occurring elastin;
a segment having at least 2 sequences, wherein each of the sequences is comprised of the three amino acid repetitive sequence found in naturally occurring collagen;
a segment having at least 2 sequences, or wherein each of the sequences is comprised of the seven amino acid repetitive sequence found in naturally occurring keratin; or
combinations thereof;
wherein at least one amino acid residue is replaced by a lysine, histidine, serine, aspartaic acid, glutamic acid, arginine, threonine, or cysteine residue.

5. The composition of claim 4, wherein the protein comprises at least two segments found in naturally occurring silk, and at least two segments found in naturally occurring elastin.

6. The composition of claim 4, wherein the sequence of amino acids found in naturally occurring silk (Sk) is GlyAlaGlyAlaGlySer.

7. The composition of claim 4, wherein the sequence of amino acids found in naturally occurring elastin (El) is GlyValGlyValPro.

8. The composition of claim 4, wherein the sequence of amino acids found in naturally occurring collagen (Cl) is GlyAlaPro.

9. The composition of claim 4, wherein the sequence of amino acids found in naturally occurring keratin (Kr) is LysLeuGluLeuAlaGluAla.

10. The composition of claim 2, wherein the protein has the formula:
NH₂—Uₙ₆—[[B]ₙ₁-[J]ₙ₂]ₙ₃-Z—COOH
wherein:
U and Z represent amino acid sequences useful in cloning the protein;
B independently at each position represents Sk, Es, Cl, or Kr;
J independently at each position represents Sk, Es, Cl, or Kr, but not the sequence in B;
Sk represents GlyAlaGlyAlaGlySer;
Es represents GlyValGlyValPro;
Cl represents GlyAlaPro;
Kr represents LysLeuGluLeuAlaGluAla;
n1 represents 1-12;
n2 represents 1-16;
n3 represents 1-30;
n6 and n7 independently represent 0 or 1 and
wherein at least one of the sequences represented by B or J is modified to comprise at least one lysine, histidine, serine, aspartaic acid, glutamic acid, arginine, threonine, cysteine residue.

11. The composition of claim 10, wherein B represents Sk or Es.

12. The composition of claim 11, wherein J represents Sk or Es.

13. The composition of claim 11, wherein J represents Es.

14. The composition of claim 10, wherein the protein has the formula:
NH₂—(U)ₙ₆-[(Sk)ₙ₃(Es)ₙ₄]ₙ₅—(Z)ₙ₇—COOH
wherein:
n3 represents 1-12;
n4 represents 1-16;
n5 represents 1-30; and
n6 and n7 independently represent 0 or 1.
wherein at least one of the sequences represented by Sk or Es is modified to comprise at least one lysine, histidine, serine, aspartaic acid, glutamic acid, arginine, threonine, cysteine residue.

15. The composition of claim 14, wherein the protein has the formula:
NH₂—(U)ₙ₆-[(Sk)₂(Es)₈]₁₇—(Z)ₙ₇—COOH
wherein at least one of the sequences represented by Sk or Es is modified to comprise at least one lysine, histidine, serine, aspartaic acid, glutamic acid, arginine, threonine, cysteine residue.

16. The composition of claim 15, wherein at least one Es segment is replaced by an amino acid sequence selected from the group consisting of GlyLysGlyValPro or GlyValGlyLysPro

17. The composition of claim 14, wherein the protein has the formula:
NH₂—(U)ₙ₆-[(Sk)₂(Es)₄Es′(Es)₃]₁₇—(Z)ₙ₇—COOH
wherein Es′ represents GlyLysGlyValPro or GlyValGlyLysPro

18. The composition of claim 17, wherein Es′ represents GlyLysGlyValPro (P27K), and wherein n6 and n7 represent 1.

19. The composition of claim 18, wherein the protein has an amino acid sequence that is at least 95% identical to the amino acid sequence of P27K.

20. The composition of claim 2, wherein the metal contrast agent comprises a metal in powder or salt form.

21. The composition of claim 20, wherein the metal contrast agent comprises a metal in salt form selected from the group consisting of an oxide, carbonate, sulfate, fluoride, chloride, and bromide salt of a metal selected from the group consisting of tantalum, tungsten, titanium, gold, platinum, iridium, rhodium, palladium, barium, gadolinium, zirconium, and bismuth.

22. The composition of claim 20, wherein the metal contrast agent is zirconium oxide; barium sulfate; bismuth oxide, or bismuth subcarbonate.

23. The composition of claim 2, wherein the crosslinker has the formula: X¹—R—X²; wherein X¹ and X² are groups that form covalent bonds with NH₂ or OH groups, and R represents a saturated or unsaturated hydrocarbyl chain having a minimum of 2, preferably a minimum of 3, and more preferably a minimum of 4 carbon atoms in the chain; and a maximum of 24, preferably a maximum of 12, and more preferably a maximum of 8. The carbon atoms in the hydrocarbyl chain may optionally be replaced by at least one heteroatom; wherein heteroatoms are selected from the group consisting of —O— or —NH₂—; and wherein each heteroatom is separated from each other heteroatom by at least two carbon atoms.

24. The composition of claim 23, wherein X¹ and X² are ester groups or isocyanate groups.

25. The composition of claim 2, wherein the crosslinker is selected from the group consisting of tetramethylene diisocyanate, hexamethylene diisocyanate, octamethylene diisocyanate, tolylene diisocyanate, diphenylmethane diisocyanate, glutaraldehyde, succinic acid dianhydride, ethylene diamine tetraacetic acid dianhydride, bis(sulfosuccinimidyl)suberate, ethyleneglycol bis(sulfosuccinimidyl)succinate, disulfosuccinimidyl tartrate, EDC (1-ethyl-3-[3-dimethylaminopropyl]carbodiimide, sulfosuccinic acid and combinations thereof.

26. The composition of claim 2, wherein the crosslinker is hexamethylene diisocyanate.

27. A method for preparing a hydrogel suitable for use as replacement material for all or part of a disc nucleus, the method comprising:
(a) mixing:
(1) an aqueous solution of an injectable, curable, cross-linkable, non-immunogenic, non-toxic protein that has at least one crosslinkable amino acid;
(2) a powder or suspension comprising a metal contrast agent in an amount sufficient to cause the hydrogel to have discernable radiopacity; and
(3) a crosslinker; and
(b) curing the composition
wherein:
(i) the hydrogel has a static compressive modulus of at least about 10 kPa and a dynamic compressive modulus of at least about 10 kPa;
(ii) the static and/or dynamic compressive modulus of the hydrogel may be lower than the static and/or dynamic compressive modulus would have been had the powder or suspension comprising a metal contrast agent not been present in the aqueous solution of the protein; and
(iii) the amount of the crosslinker is sufficient to restore, on curing, at least about 100% of the decrease in static and/or dynamic compressive modulus of the hydrogel caused by the presence of the powder or suspension comprising a metal contrast agent in the aqueous solution of the protein.

28. The composition of claim 2, wherein the metal contrast agent comprises a metal in powder or salt form.

29. The composition of claim 20, wherein the metal contrast agent comprises a metal in salt form selected from the group consisting of an oxide, carbonate, sulfate, fluoride, chloride, and bromide salt of a metal selected from the group consisting of tantalum, tungsten, titanium, gold, platinum, iridium, rhodium, palladium, barium, gadolinium, zirconium, and bismuth.

30. The composition of claim 20, wherein the metal contrast agent is zirconium oxide; barium sulfate; bismuth oxide, or bismuth subcarbonate.

31. A hydrogel suitable for use as replacement material for all or part of a disc nucleus wherein the hydrogel is prepared by curing the composition of claim 1.

32. A hydrogel suitable for use as replacement material for all or part of a disc nucleus wherein the hydrogel is prepared by curing the composition of claim 2.

33. A method for replacing a disc nucleus in a human patient in need thereof, the method comprising:
(a) preparing a liquid composition by mixing:
(1) an aqueous solution of an injectable, curable, cross-linkable, non-immunogenic, non-toxic protein that has at least one crosslinkable amino acid;
(2) a powder or suspension comprising a metal contrast agent in an amount sufficient to cause the hydrogel to have discernable radiopacity; and
(3) a crosslinker;
(b) injecting the liquid composition into a disc of the human patient; and
(c) curing the composition under conditions that cause the liquid composition to become a hydrogel in the disc of the human patient;
wherein:
(i) the hydrogel has a static compressive modulus of at least about 10 kPa and a dynamic compressive modulus of at least about 10 kPa;
(ii) the static and/or dynamic compressive modulus of the hydrogel may be lower than the static and/or dynamic compressive modulus would have been had the powder or suspension comprising a metal contrast agent not been present in the aqueous solution of the protein; and
(iii) the amount of the crosslinker is sufficient to restore, on curing, at least about 100% of the decrease in static and/or dynamic compressive modulus of the hydrogel caused by the presence of the powder or suspension comprising a metal contrast agent in the aqueous solution of the protein.

34. A kit comprising:
(a) a container comprising a minimum of about 10% and a maximum of about 25% of a curable, cross-linkable, non-immunogenic, non-toxic protein;
(b) a container comprising a minimum of about 2% and a maximum of about 70% of a metal contrast agent; and
(c) a container comprising a minimum of about 0.1% and a maximum of about 10% of a crosslinker;
wherein:
a liquid composition is formed upon mixing the contents of container (a), container (b) and container (c);
the liquid composition undergoes curing and becomes a hydrogel; and
the hydrogel has a discernable radiopacity level.

35. A kit comprising:
(a) an aqueous solution of a curable, cross-linkable, non-immunogenic, non-toxic protein containing at least one crosslinkable amino acid;
(b) a powder or suspension comprising a metal contrast agent in an amount sufficient to cause the hydrogel to have discernable radiopacity; and
(c) a crosslinker;
wherein:
a liquid composition is formed upon mixing the contents of container (a), container (b) and container (c);
the liquid composition undergoes curing and becomes a hydrogel;
the hydrogel has a discernable radiopacity level;
the hydrogel has a static compressive modulus of at least about 10 kPa and a dynamic compressive modulus of at least about 10 kPa;
the static and/or dynamic compressive modulus of the hydrogel may be lower than the static and/or dynamic compressive modulus would have been had the powder or suspension comprising a metal contrast agent not been present in the aqueous solution of the protein; and
the amount of the crosslinker is sufficient to restore at least about 100% of the decrease in static and/or dynamic compressive modulus of the hydrogel caused by the presence of the powder or suspension comprising a metal contrast agent in the aqueous solution of the protein.
